(19) United States
(12) Patent Application Publication
(10) Pub. No.: US 2026/0335300 A1
(43) Pub. Date: Oct. 29, 2026

(54) Synthetic Nicotine Analogue Formulation For Nicotine Replacement and Amplication

(71) Applicant: Purple Sky, LLC, Cheyenne, WY (US)

(72) Inventors: Micheal Kriznic, Trieste (IT); Samuel Benaim, Cheyenne, WY (US)

(21) Appl. No.: 19/486,013

(22) PCT Filed: May 22, 2024
(86) PCT No.: PCT/US2024/030538

§ 371 Date: Nov. 19, 2025

Related U.S. Application Data
(60) Provisional application No. 63/468,343, filed on May 23, 2023.
(60) Provisional application No. 63/468,351, filed on May 23, 2023.
(60) Provisional application No. 63/468,349, filed on May 23, 2023.

(51) Int. Cl. A61K 31/465; A24B 15/167; C07D 401/04

(52) U.S. Cl. CPC A61K 31/465; A24B 15/167; C07D 401/04

(57) ABSTRACT

A synthetic nicotine replacement for various delivery systems, particularly for use in electronic nicotine delivery systems, for example, vaporizers, e-cigarettes, combustion, heat-not-burn products, oral, or nasal nicotine delivery systems. (S)-6-Methylnicotine, also known as (S)-2-methyl-5-(1-methylpyrrolidin-2-yl)pyridine, is a derivative of nicotine with a methyl substitution at the 6-position of the pyridine ring. Preliminary empirical data indicates that (S)-6-Methylnicotine is approximately 4 to 5 times more potent than nicotine per unit of mass, and may be faster acting, providing a more effective solution for managing or satisfying nicotine cravings. Carboxylate salts thereof are also suitable. This increased strength allows individuals to experience relief from nicotine cravings more quickly and effectively, using much lower dosages, and induces a subjective satisfaction sensation which satisfies cravings. In addition to other potential benefits, this increased pharmacological potency allows for the use of a reduced amount of active ingredient when compared to synthetic nicotine formulations.

### FIELD

Certain aspects of the present disclosure generally relate to a synthetic nicotine replacement for electronic delivery systems, particularly for use in vaporizers and heat-not-burn delivery systems.

### BACKGROUND

Nicotine is a highly addictive substance that is commonly used as a stimulant and is found in various forms, including tobacco products, nicotine replacement therapy products, and electronic nicotine delivery systems. Despite its widespread use, nicotine is associated with negative health effects, including increased risk of heart disease, stroke, and various types of cancer.

During the early 1990s, the World Health Organization fervently promoted nicotine replacement therapy on a global scale, a method aimed at alleviating the physiological and psychological dependencies incited by the combustion of conventional cigarettes. This move marked the inception of a burgeoning new tobacco industry that developed innovative products like atomized electronic cigarettes and heat-not-burn tobacco products as a preferred alternative to conventional smoking habits.

Atomized electronic cigarettes in current markets are typically synthesized from a concoction of propylene glycol, glycerol, flavorings, and nicotine salts. The heat-not-burn products utilize tobacco or non-tobacco herbal particulates as carriers. These carriers incorporate typical additives such as nicotine, flavorings, sweetening agents, and cooling agents, dissolved in prevalent solvents like propylene glycol, glycerol, or water. This solution is homogenously combined with the carrier and encapsulated into heat-not-burn cartridges, effectively omitting tar derivatives produced through traditional cigarette combustion.

Nevertheless, the utilization of commercially available nicotine presents several considerable challenges. The principal source of such nicotine, natural levorotatory nicotine extracted from tobacco, is susceptible to a myriad of influential factors including variance in tobacco crop yield and climate change. Additionally, it is subject to alterations instigated by tobacco-related policies, making it unsuitable for extensive application. The artificial synthesis of nicotine yields racemic nicotine, comprising R and S configurations. However, when incorporated in novel tobacco products, this form of nicotine does not induce the distinctive satisfaction and ‘throat-hit’ sensation pursued by smokers. This necessitates the process of chiral separation, a procedure associated with substantial costs.

Consequently, researchers in the emergent tobacco industry are investigating the potential of alkaloids as alternatives to nicotine. Conventional alkaloids, including cytisine and arecoline, have been examined for this purpose. However, their incorporation into novel tobacco products has been met with disappointment due to the lack of satisfaction and ‘throat-hit’ sensation, culminating in an inferior sensory experience for consumers. Thus, the quest for a gratifying and effective nicotine substitute or nicotine enhancing agent remains a prominent challenge within this rapidly advancing field.

Nicotine is absorbed into the bloodstream through the mucous membranes in the mouth and lungs. The speed and efficiency of nicotine absorption can vary depending on the method of delivery, such as smoking, chewing, or using nicotine replacement therapy products such as patches, gum, or lozenges.

When a person smokes tobacco, nicotine is rapidly absorbed into the bloodstream through the lungs and reaches the brain within seconds. This quick delivery of nicotine is part of what makes smoking so addictive.

When a person uses a nicotine replacement therapy product, the rate of nicotine absorption is typically slower and more controlled, as the nicotine is absorbed gradually through the mucous membranes in the mouth or skin. This can help to reduce the risk of nicotine overdose and decrease the likelihood of developing dependence on nicotine.

Once in the bloodstream, nicotine is quickly distributed to various parts of the body, including the brain, where it acts as a stimulant and affects the release of certain neurotransmitters, such as dopamine and acetylcholine. The effects of nicotine can last for several hours, depending on the dose and individual metabolism.

Nicotine acts on specific receptors in the body known as nicotinic acetylcholine receptors (nAChRs). These receptors are found in various parts of the body, including the central and peripheral nervous systems, and they play a role in various physiological processes such as autonomic ganglionic transmission, muscle contraction, and synaptic transmission in the brain.

There are several subtypes of nAChRs, each with a different distribution and function. Nicotine primarily binds to alpha4beta2 nAChRs, which are found in high concentrations in the brain and are involved in the regulation of dopamine release, which is the key neurotransmitter responsible for the reinforcing effects of nicotine.

The binding of nicotine to nAChRs leads to the activation of these receptors and the release of neurotransmitters, which in turn can result in the characteristic effects of nicotine, such as increased alertness, decreased appetite, and improved cognitive function.

However, it is important to note that the chronic use of nicotine can lead to the development of tolerance and dependence, and the repeated activation of nAChRs by nicotine can cause structural and functional changes in the brain that can result in addiction.

Patent application CN114437025A discloses a method for making a racemic 6-Methylnicotine, which results in the production of a chiral compound which is a racemic mixture.

Nicotine replacement therapy products involve the controlled delivery of nicotine in order to satisfy cravings and gradually reduce dependence in nicotine cessation. However, due to regulatory limitations and costs, the amount and rate of nicotine delivered through these systems are often insufficient to satisfy nicotine cravings, thereby reducing the effectiveness of the nicotine replacement therapy. It is thus desired to provide a nicotine amplifying formulation to satisfy nicotine cravings with less nicotine.

One form of nicotine replacement therapy involves the use of electronic nicotine delivery systems, such as vaporizers or e-cigarettes. These products have attracted considerable attention in view of certain public health events in recent years. However, due to regulatory limitations and costs, the amount and rate of nicotine delivered through these systems are often insufficient to satisfy nicotine cravings, thereby reducing the effectiveness of the nicotine replacement therapy. It is thus desired to provide a synthetic nicotine analogue formulation for electronic delivery systems which offers a more effective alternative for managing nicotine cravings.

Another form of nicotine replacement therapy involves the use of oral nicotine delivery systems, such as pouches or gums. Another form of nicotine replacement therapy involves the use of nasal sprays or powders. These products have attracted considerable attention in view of certain public health events in recent years. However, due to regulatory limitations and costs, the amount and rate of nicotine delivered through these systems are often insufficient to satisfy nicotine cravings, thereby reducing the effectiveness of the nicotine replacement therapy.

As a result, researchers in the evolving oral and nasal nicotine industry are exploring the use of alkaloids as potential nicotine replacements. Conventional alkaloids, such as cytisine and arecoline, have been considered. However, their introduction into novel oral and nasal nicotine products has been met with limited satisfaction due to a lack of satisfactory sensations, resulting in an inferior sensorial experience for consumers. Thus, the pursuit of a satisfying and effective nicotine substitute for oral and nasal products remains a significant challenge in this rapidly advancing field. It is thus desired to provide a synthetic nicotine analogue formulation for oral and nasal delivery systems which offers a more effective alternative for managing or satisfying nicotine cravings and provides potentially reduced toxicity and potentially increased product stability.

### SUMMARY

Without limiting the scope of the appended claims, some prominent features are described herein.

Details of one or more implementations of the subject matter described in this specification are set forth in the accompanying drawings and the description below. Other features, aspects, and advantages will become apparent from the description, the drawings, and the claims. Note that the relative dimensions of the following figures may not be drawn to scale.

One aspect of the present disclosure provides a use of a synthetic nicotine analogue compound, (S)-6-Methylnicotine as a nicotine replacement in electronic nicotine delivery systems, for example, vaporizers, e-cigarettes or heat-not-burn products to provide improved effectiveness, safety and cost reductions over existing devices.

One aspect of the present disclosure provides a use of a synthetic nicotine analogue compound, S-(6)-Methylnicotine as a nicotine replacement in oral or nasal nicotine delivery systems for example, nicotine pouches or gums to provide improved effectiveness, safety and cost reductions over existing devices.

One aspect of the present disclosure provides a use of a synthetic nicotine analogue compound, S-(6)-Methylnicotine as a nicotine amplifying agent to provide improved effectiveness, safety and cost reductions over existing devices.

One aspect of the present disclosure provides a use of a synthetic nicotine analogue as a nicotine replacement, wherein the synthetic nicotine analogue is at least one of (S)-6-methylnicotine or a (S)-6-methylnicotine carboxylate salt.

The (S)-6-methylnicotine carboxylate salt may be an acetate, ascorbate, benzoate, butyrate, citrate, lactate, levulinate, malate, pyruvate, succinate, tartrate, or salicylate salt, or any other commercially and organoleptically viable carboxylate salt. The synthetic nicotine analogue can be used as a nicotine replacement for electronic delivery systems. The synthetic nicotine analogue can be used as a nicotine replacement in combustion applications. The synthetic nicotine analogue can be used as a nicotine replacement in heat-not-burn applications. The synthetic nicotine analogue can be used as a nicotine replacement in oral delivery applications. The synthetic nicotine analogue can be used as a nicotine replacement in nasal delivery applications. The synthetic nicotine analogue can be used as a nicotine replacement in heat-not-burn applications. The synthetic nicotine analogue can also be used for nicotine replacement therapy.

Another aspect of the present disclosure provides a use of a synthetic nicotine analogue as a nicotine amplification agent, wherein the synthetic nicotine analogue is at least one of(S)-6-methylnicotine or a (S)-6-methylnicotine carboxylate salt. The (S)-6-methylnicotine carboxylate salt may be an acetate, ascorbate, benzoate, butyrate, citrate, lactate, levulinate, malate, pyruvate, succinate, tartrate, or salicylate salt.

Yet another aspect of the present disclosure provides a synthetic nicotine analogue comprising a (S)-6-methylnicotine or a (S)-6-methylnicotine carboxylate salt. The (S)-6-methylnicotine carboxylate salt may be an acetate, ascorbate, benzoate, butyrate, citrate, lactate, levulinate, malate, pyruvate, succinate, tartrate, or salicylate salt.

### BRIEF DESCRIPTION OF THE DRAWINGS

FIG. 1 is the chemical structure of (S)-6-Methylnicotine also known as (S)-2-methyl-5-(1-methylpyrrolidin-2-yl)pyridine.

FIG. 2 is a table showing study results comparing the biological effect of the formulation compared to nicotine according to one aspect of the present disclosure.

FIG. 3 is a table showing preliminary acute oral toxicity test results in mice for (S)-6-Methylnicotine according to one aspect of the present disclosure.

FIG. 4 is a table showing preliminary acute oral toxicity test results for (S)-6-Methylnicotine benzoate according to one aspect of the present disclosure.

FIG. 5 is a reaction scheme showing synthesis of (S)-6-Methylnicotine acetate.

FIG. 6 is a reaction scheme showing synthesis of (S)-6-Methylnicotine ascorbate.

FIG. 7 is a reaction scheme showing synthesis of (S)-6-Methylnicotine benzoate.

FIG. 8 is a reaction scheme showing synthesis of (S)-6-Methylnicotine butyrate.

FIG. 9 is a reaction scheme showing synthesis of (S)-6-Methylnicotine citrate.

FIG. 10 is a reaction scheme showing synthesis of (S)-6-Methylnicotine lactate.

FIG. 11 is a reaction scheme showing synthesis of (S)-6-Methylnicotine levulinate.

FIG. 12 is a reaction scheme showing synthesis of (S)-6-Methylnicotine malate.

FIG. 13 is a reaction scheme showing synthesis of (S)-6-Methylnicotine pyruvate.

FIG. 14 is a reaction scheme showing synthesis of (S)-6-Methylnicotine succinate.

FIG. 15 is a reaction scheme showing synthesis of (S)-6-Methylnicotine tartrate.

FIG. 16 is a reaction scheme showing synthesis of (S)-6-Methylnicotine salicylate.

### DETAILED DESCRIPTION

Various aspects of the novel systems, apparatuses, and methods are described more fully hereinafter with reference to the accompanying drawings. The teachings disclosure can, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the invention. For example, an apparatus can be implemented, or a method can be practiced using any number of the aspects set forth herein. In addition, the scope of the invention is intended to cover such an apparatus or method which is practiced using other structure, functionality, or structure and functionality in addition to or other than the various aspects of the invention set forth herein. Any aspect disclosed herein can be embodied by one or more elements of a claim.

Although aspects are described herein, many variations and permutations of these aspects fall within the scope of the disclosure. Although some benefits and advantages of the preferred aspects are mentioned, the scope of the disclosure is not intended to be limited to benefits, uses, or objectives. The detailed description and drawings are merely illustrative of the disclosure rather than limiting.

In accordance with one disclosed aspect, the present invention provides a use of a synthetic nicotine analogue formulation in electronic delivery systems for nicotine replacement therapy.

In accordance with one disclosed aspect, the present invention provides a use of a synthetic nicotine analogue formulation in electronic delivery systems.

In accordance with one disclosed aspect, the present invention provides a use of a synthetic nicotine analogue formulation in heat-not-burn delivery systems for nicotine replacement therapy.

In accordance with one disclosed aspect, the present invention provides a use of a synthetic nicotine analogue formulation in heat-not-burn delivery systems.

In accordance with one disclosed aspect, the present invention provides a use of a synthetic nicotine analogue formulation in oral delivery systems for nicotine replacement therapy.

In accordance with one disclosed aspect, the present invention provides a use of a synthetic nicotine analogue formulation in nasal delivery systems for nicotine replacement therapy.

In accordance with one disclosed aspect, the present invention provides a use of a synthetic nicotine analogue formulation in oral delivery systems to provide a delivery method that provides enjoyment and satisfaction to a user that is similar to the effects provided by nicotine.

In accordance with one disclosed aspect, the present invention provides a use of a synthetic nicotine analogue formulation in nasal delivery systems to provide a delivery method that provides enjoyment and satisfaction to a user that is similar to the effects provided by nicotine.

In accordance with one disclosed aspect, the present invention provides a use of a synthetic nicotine analogue formulation for use as a nicotine amplifier for nicotine replacement therapy.

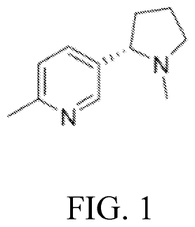

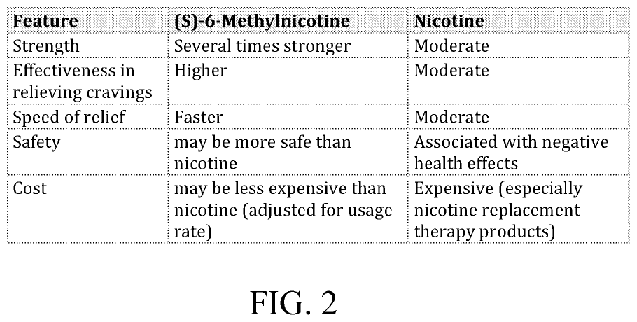

FIG. 1 is an illustration of the chemical structure of a preferred embodiment of a synthetic nicotine analogue, (S)-6-Methylnicotine, also known as (S)-2-methyl-5-(1-methylpyrrolidin-2-yl)pyridine. 6-Methylnicotine is a derivative of nicotine with a methyl substitution at the 6-position of the pyridine ring. 6-Methylnicotine has been used as a precursor in the preparation of other 6-subtituted nicotine derivatives.

6-Methylnicotine is a chiral compound and is typically produced in a racemic mixture. In the racemate, the efficacy of 6-Methylnicotine is reduced, as the R-enantiomer, (R)-6-Methylnicotine is not effective as a nicotine analogue, and contributes to unpleasant flavors, and is an undesirable waste product which dilutes the effectiveness and potentially increases the hazardous side effects. In an embodiment, the synthetic nicotine analogue formulation comprises between 99% and 100%, preferably between 99.5% and 100%, and ideally between 99.8% and 100% enantiomerically pure (S)-6-Methylnicotine.

In one embodiment, a synthetic nicotine analogue formulation may be adapted for use for electronic nicotine delivery systems, such as vaporizers or e-cigarettes. The formulation is designed to be used in existing electronic nicotine delivery systems without need for modification. The formulation contains between 0.01% and 99.5% (S)-6-Methylnicotine, preferably between 0.20% and 10.00% and optimally between 0.50% and 2.00%. The formulation may additionally comprise methyl cellulose or other similar thickeners, humectants, flavors, and other active ingredients such as caffeine, nicotine, taurine, or guaranine, for example. The formulation has a pH between 5 and 10.5, preferably between 7 and 10, and optimally between 8.0 and 9.5. In some embodiments, the formulation comprises PG/VG (propylene glycol and vegetable glycerin). In some embodiments, the formulation further comprises flavoring in liquid form.

In one exemplary embodiment, (S)-6-Methylnicotine is used instead of nicotine in an e-liquid for use in nicotine replacement therapy.

In another exemplary embodiment, (S)-6-Methylnicotine is used instead of nicotine in an e-liquid for use in electronic nicotine delivery systems.

In another exemplary embodiment, (S)-6-Methylnicotine is used instead of nicotine in a heat-not-burn carrier materials for use in nicotine replacement therapy.

In another exemplary embodiment, (S)-6-Methylnicotine is used instead of nicotine in a heat-not-burn carrier materials for use in heat-not-burn delivery systems.

In an embodiment of a synthetic nicotine analogue formulation, it may be adapted for use for oral nicotine delivery systems, such as nicotine pouches, chewing gum, strips, lozenges, tablets, gummies, gels, thin films, or other forms designed to be placed inside a user's mouth. The formulation is designed to be used in existing oral nicotine delivery systems without need for extensive modification. The formulation contains between 0.01% and 99.5% (S)-6-Methylnicotine, preferably between 0.01% and 30.0% and optimally between 1.00% and 18.00%. The formulation may additionally comprise methyl cellulose or other similar thickeners, humectants, flavors, and other active ingredients such as caffeine, nicotine, taurine, or guaranine, for example. The formulation has a pH between 5 and 10.5, preferably between 7 and 10, and optimally between 8.0 and 9.5. The moisture level of the formulation is below 50%, and preferably between 15% and 35%. In some embodiments, (S)-6-Methylnicotine is bound with a salt, polymer, or other ingredient including but not limited to: tartaric acid, benzoic acid, lactic acid, malic acid, polacrilex resin, or betacylcodextrin. In some embodiments, the formulation is processed using a high sheer mixer to increase potential uptake. In yet other embodiments, (S)-6-Methylnicotine may be combined with a lipid or a combination of lipids, such as monoglyceride or a triglyceride, to reduce bitterness, improve texture, facilitate absorption, provide stability or increased shelf-life, or to modify the release profiles of the (S)-6-Methylnicotine and/or flavourant(s) and/or sweetener(s), thereby increasing the user experience. In some embodiments, the formulation further comprises one of more flavorings in liquid or solid form.

In an embodiment of a synthetic nicotine analogue formulation, it may be adapted for use for nasal nicotine delivery systems, such sprays, powders, liquids, gels or other forms designed to be delivered inside a user's nasal cavity. The formulation is designed to be used in existing nasal nicotine delivery systems without need for extensive modification. The formulation contains between 0.01% and 99.5% (S)-6-Methylnicotine, preferably between 0.01% and 30.0% and optimally between 1.00% and 18.00%. The formulation may additionally comprise moisturizers or other similar oils, humectants, flavors, and other active ingredients such as caffeine, nicotine, taurine, or guaranine, for example. The formulation has a pH between 5 and 10.5, preferably between 7 and 10, and optimally between 8.0 and 9.5. In some embodiments, (S)-6-Methylnicotine is bound with a salt, polymer, or other ingredient including but not limited to: tartaric acid, benzoic acid, lactic acid, malic acid, polacrilex resin, or betacylcodextrin. In some embodiments, the formulation is processed using a high sheer mixer to increase potential uptake. In yet other embodiments, (S)-6-Methylnicotine may be combined with a lipid or a combination of lipids, such as monoglyceride or a triglyceride, to reduce bitterness, improve texture, facilitate absorption, provide stability or increased shelf-life, or to modify the release profiles of the (S)-6-Methylnicotine and/or flavourant(s) and/or sweetener(s), thereby increasing the user experience. In some embodiments, the formulation further comprises one of more flavorings in liquid or solid form. In some embodiments, the formulation further comprises one of more additional active ingredients in liquid or solid form.

In one exemplary embodiment, (S)-6-Methylnicotine is used instead of nicotine in a nicotine pouch for use in nicotine replacement therapy.

In another exemplary embodiment, (S)-6-Methylnicotine is used instead of nicotine in a nicotine gum for use in nicotine replacement therapy.

In one exemplary embodiment, (S)-6-Methylnicotine is used instead of nicotine in a nicotine spray for use in nicotine replacement therapy.

In another exemplary embodiment, (S)-6-Methylnicotine is used instead of nicotine in a nicotine powder for use in nicotine replacement therapy.

In one exemplary embodiment, (S)-6-Methylnicotine is used instead of nicotine in a nicotine pouch for use in nicotine replacement products.

In another exemplary embodiment, (S)-6-Methylnicotine is used instead of nicotine in a nicotine gum for use in nicotine replacement products.

In one exemplary embodiment, (S)-6-Methylnicotine is used instead of nicotine in a nicotine spray for use in nicotine replacement products.

In another exemplary embodiment, (S)-6-Methylnicotine is used instead of nicotine in a nicotine powder for use in nicotine replacement products.

Preliminary empirical data indicates that (S)-6-Methylnicotine is approximately 4 to 5 times more potent than nicotine per unit mass, and may be faster acting, providing a more effective solution for managing nicotine cravings. In certain applications, preliminary results indicate that the potency may be even higher, between 6 to 8 times more potent, or even 10 to 12 times more potent. This increased strength allows individuals to experience relief from nicotine cravings more quickly and effectively, using much lower dosages, and induces a subjective “hit” sensation which satisfies cravings. This increased pharmacological potency allows for the use of a reduced amount of active ingredient when compared to synthetic nicotine formulations. As less active ingredient is required, additional inert stabilizing agents can be included in the formulation, increasing stability. Furthermore, the adverse impact of any potential impurities or decomposition products of the active ingredient, such as discoloration, degradation of taste, or other undesirable impacts are minimized due to the reduced concentrations of active ingredient required. Additionally, the relative cost of production compared to effective dosage results in cost savings when compared to traditional nicotine. Furthermore, as cravings can be satisfied using a lower amount of active ingredient, users of (S)-6-Methylnicotine based nicotine replacement therapy can require less material overall, which can thus reduce potential negative health outcomes associated with side effects caused by non-active ingredients.

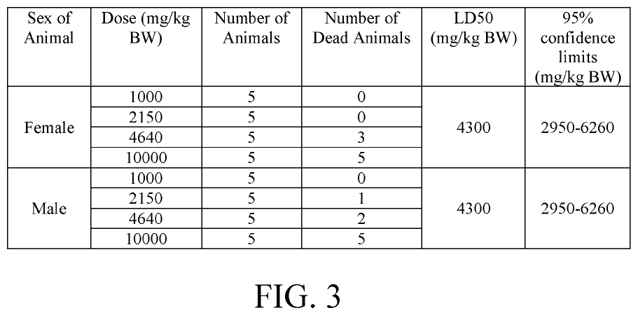

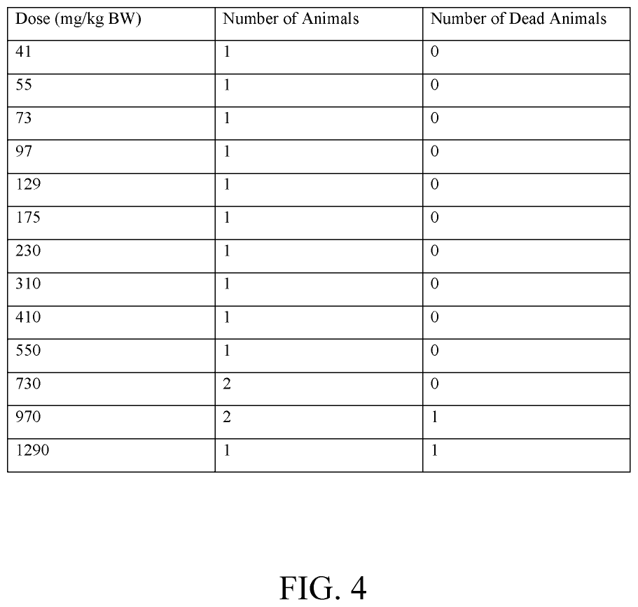

Preliminary data also appears to show that (S)-6-Methylnicotine may be less harmful than nicotine when compared to effective dose, as illustrated in the table of FIG. 3, which shows the acute oral LD₅₀ of (S)-6-Methylnicotine in mice to be between 2650 and 6980 mg/kg body weight for a 95% confidence interval, the mean being 4300 mg/kg body weight, as compared to about 3.3 mg/kg BW for nicotine, reflecting a potential three (3) order of magnitude reduction of oral toxicity in mice. A further acute oral toxicity study was conducted, using equivalent-effect diluted (S)-6-Methylnicotine benzoate in glycerol, with the active ingredient diluted to 166 mg/ml (an approximately 6:1 dilution). The diluted mixture was then administered in increasing quantities to rats. The acute oral LD₅₀ of diluted (S)-6-Methylnicotine benzoate was determined to be between 730 mg/kg and 1290 mg/kg body weight, as illustrated in the table of FIG. 4. Accounting for the dilution factor, the adjusted values of between 120 mg/kg and 215 mg/kg acute oral LD₅₀ reflects that (S)-6-Methylnicotine benzoate is half to one quarter as toxic to rats compared to nicotine, which has an acute oral LD₅₀ of 50 mg/kg for rats. On an equivalent effective use basis, these results reflect a two (2) order of magnitude reduction in toxicity. The results support finding that (S)-6-Methylnicotine, and carboxylate salts thereof, is both less toxic, and can be used in lower amounts than nicotine, resulting in significant overall reduction in toxicity.

Additionally, (S)-6-Methylnicotine has been observed to be more chemically stable than nicotine, and resists degradation and discoloration. (S)-6-Methylnicotine may potentially last up to five times (5×) longer in the air compared to nicotine which may result in an increased shelf life, and it may be able to resist higher temperatures which may also contribute to a longer shelf life and increased product stability. A test was conducted whereby (S)-6-Methylnicotine and nicotine were allowed to be exposed to atmosphere for 24 hours and nicotine yellowing was observed sooner than (S)-6-Methylnicotine, indicating that nicotine experiences more rapid decomposition that (S)-6-Methylnicotine. Test results have shown that (S)-6-Methylnicotine is more stable than nicotine when blended with other compounds, enabling combinations of active ingredients.

In some embodiments, the formulation may comprise additional secondary active ingredients in addition to (S)-6-Methylnicotine, such as Caffeine, Ethanol, Tobacco additives, and various medications, herbs and supplements. In one exemplary embodiment, Ethanol, a central nervous system depressant that can increase the absorption and potency of (S)-6-Methylnicotine leading to a stronger effect, is used to further reduce the amount of primary active ingredient required to achieve craving reduction.

In combustion and heat-not-burn applications, due to the reduced amount of (S)-6-Methylnicotine required compared to nicotine, it is preferrable to dilute the (S)-6-Methylnicotine in an excipient, such as an appropriate solvent including glycerol, propylene glycol, or water, for example. The dissolved and diluted (S)-6-Methylnicotine can then be applied to a substrate to ensure uniform and homogenous distribution across the substrate surface. Suitable substrates include natural biomaterials, such as ground herbal or botanical blends, of tobacco or non-tobacco origin, artificial or synthetic fibrous or ground materials, or any other type of natural or artificial porous material capable of adsorbing liquid or particulates, for example. In one exemplary aspect, a liquid solution containing (S)-6-Methylnicotine is atomized or aerosolized using an air-powered sprayer, such as an airbrush, and applied to an appropriate substrate. In other embodiments, the substrate may be immersed or coated with liquid solution. The concentration and amount of liquid solution applied can be finely controlled to control the amount of (S)-6-Methylnicotine present to ensure consistent dosage. Where the substrate is tobacco, or another combustible or heat-activated active plant substrate such as cannabis, (S)-6-Methylnicotine may be used as the main active compound, as or co-active compound, or as an enhancer or amplifier.

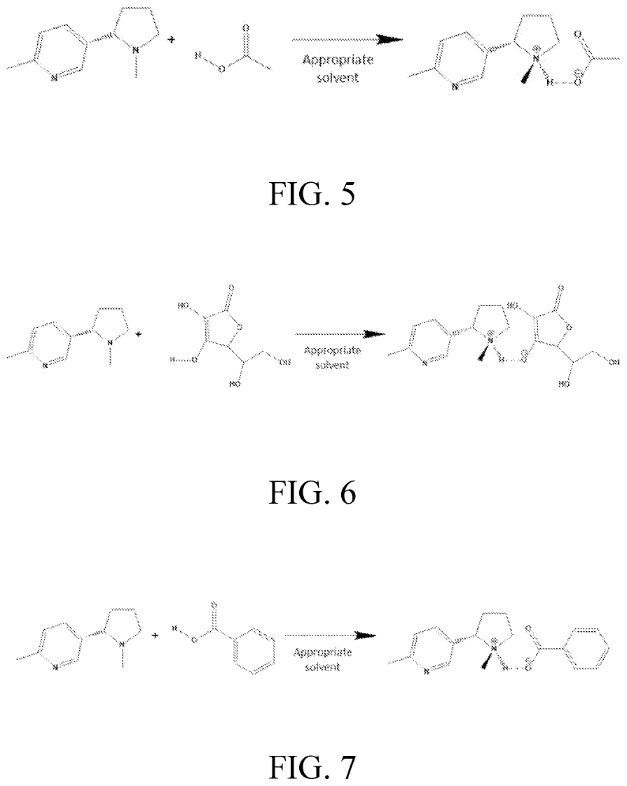

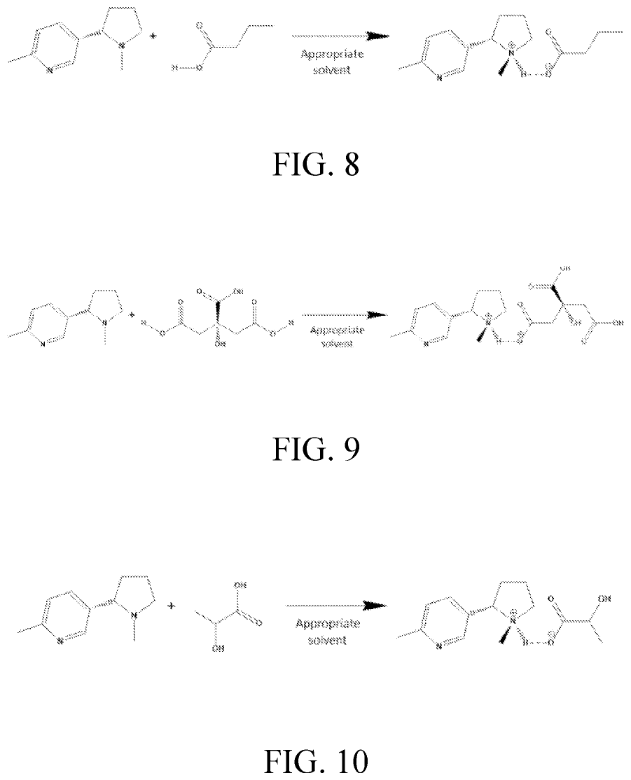

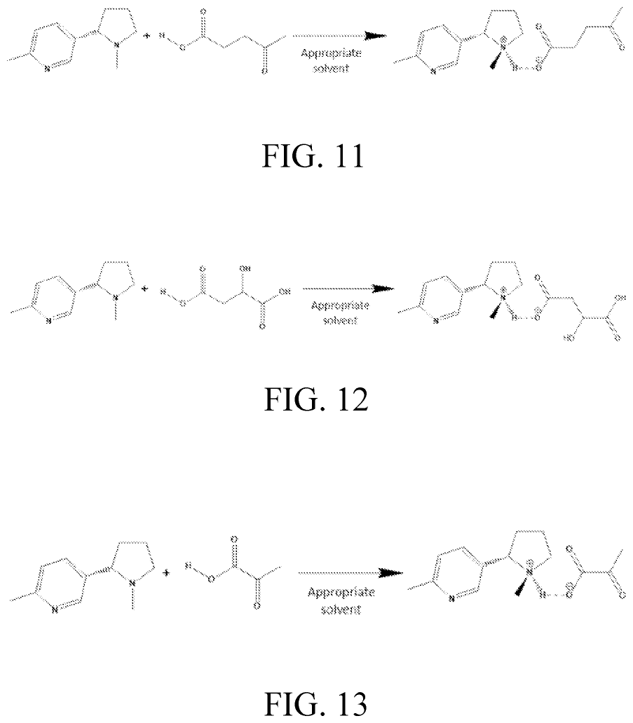

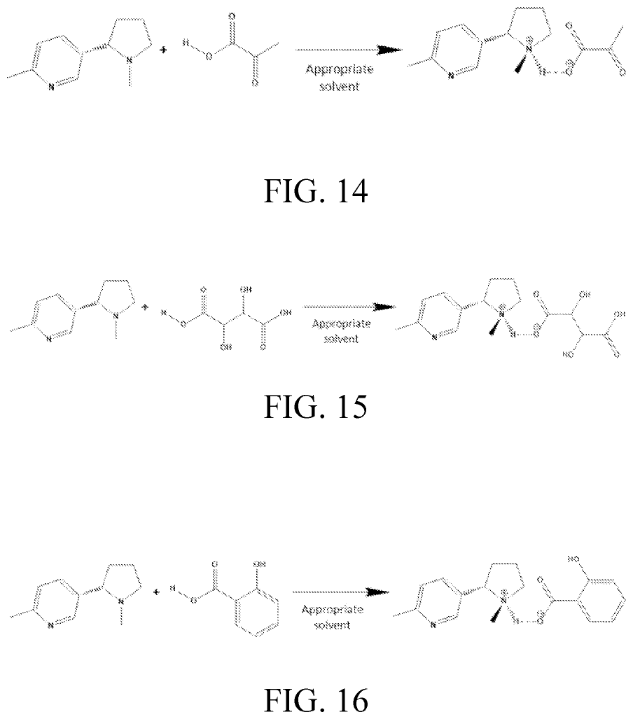

While the aforementioned embodiments refer to (S)-6-Methylnicotine, in some embodiments, (S)-6-Methylnicotine may be replaced with or used in combination with one or more (S)-6-Methylnicotine carboxylate salts. (S)-6-Methylnicotine carboxylate salts can be synthesized by reaction of (S)-6-Methylnicotine with the corresponding carboxylic acid in appropriate solvent. Appropriate solvents include common solvents such as glycerol, propylene glycol, and water, which are generally considered safe for human consumption. As shown in FIGS. 5 to 16, novel (S)-6-Methylnicotine carboxylates were synthesized and tested. (S)-6-Methylnicotine carboxylates synthesized include (S)-6-Methylnicotine acetate, (S)-6-Methylnicotine ascorbate, (S)-6-Methylnicotine benzoate, (S)-6-Methylnicotine butyrate, (S)-6-Methylnicotine citrate, (S)-6-Methylnicotine lactate, (S)-6-Methylnicotine levulinate, (S)-6-Methylnicotine malate, (S)-6-Methylnicotine pyruvate, (S)-6-Methylnicotine succinate, (S)-6-Methylnicotine tartrate, and (S)-6-Methylnicotine salicylate. While these novel variations were synthesized and tested, a similar synthesis route applies generally to any other carboxylic acid, all of which are contemplated.

Trials have shown that the use of mixtures of (S)-6-Methylnicotine, in free base form, in combination with a carboxylate salt thereof provides benefits. In some embodiments, inclusion of carboxylate salts of (S)-6-Methylnicotine can be used to modify the sensory experience. (S)-6-Methylnicotine carboxylate salts have been found to have reduced perceived ‘harshness’ or ‘burning’ sensation, and improves the perceived texture of (S)-6-Methylnicotine formulations for some delivery methods, such as vaporizer, combustion, or heat-not-burn. In yet other embodiments, the formulations may further comprise further active ingredients, such as a mild local anesthetic, which modulates the sensory experience of the surfaces of the human body, such as the nostril and nasal cavity, in order to further enhance the user experience.

The molar ratio of carboxylic acid and free base (S)-6-Methylnicotine can be adjusted to attain a more effective pH level for the respective delivery methods. Further, preliminary studies indicate that carboxylate salts of (S)-6-Methylnicotine appear to be more stable, and further improve the stability of, (S)-6-Methylnicotine, allowing for further product stability over time.

For example, for electronic delivery systems (vaporizers), the optimal ratio of carboxylic acid to free base (S)-6-Methylnicotine is between 1:1 to 1.6:1, with an equivalent amount or excess of carboxylic acid. Adjustment of the molar ratio of carboxylate salt to free base(S)-6-Methylnicotine also allows for tuning of the perceived user experience. Increasing carboxylate salt content reduces the instantaneous “throat hit” sensation, and smooths the sensation. Furthermore, the carboxylic acid additional imparts flavor, and can be used to compliment the flavoring of the e-liquid. Preferred carboxylates for the vaporizer application include (S)-6-Methylnicotine benzoate, (S)-6-Methylnicotine lactate, (S)-6-Methylnicotine levulinate, and (S)-6-Methylnicotine salicylate. (S)-6-Methylnicotine levulinate is noted for having a particularly desirable flavor in mixture. Other carboxylates may offer superior texture and sensation, but may have corresponding tradeoffs in terms of flavor.

Conversely, for oral applications, the preferred pH is basic, and accordingly, the preferred ratio of carboxylic acid to free base (S)-6-Methylnicotine is less than or equal to 1:1, with an excess or equivalent amount of free base (S)-6-Methylnicotine. While it is possible to have excess carboxylic acid, the resulting pH-lowering due to the present of acid requires additional buffering agents.

For vaporizer and combustion applications, the carboxylate salt form has superior organoleptic properties, with the sensation being described as less harsh and peppery, and smoother than the free base (S)-6-Methylnicotine. Similar user experiences have been reported for both oral and nasal application of the (S)-6-Methylnicotine carboxylate salt compared to the free base. In such applications, the texture effect of the carboxylate is the dominant consideration over flavor, as the combustion or partial-combustion process releases a degree of smoke, which masks the flavor of the carboxylates.

Furthermore (S)-6-Methylnicotine carboxylate salts offer superior solubility, in the appropriate solvents, compared to free base (S)-6-Methylnicotine, though the solubility of free base (S)-6-Methylnicotine has been found to be acceptable for all of the aforementioned applications.

While the foregoing is directed to aspects of the present disclosure, other and further aspects of the disclosure can be devised without departing from the basic scope thereof.

1. Use of a synthetic nicotine analogue as a nicotine replacement, wherein the synthetic nicotine analogue comprises enantiomerically pure (S)-6-methylnicotine or a carboxylate salt thereof.

2. The use of claim 1, wherein the synthetic nicotine analogue comprises a (S)-6-methylnicotine carboxylate salt.

3. The use of claim 2, wherein the (S)-6-methylnicotine carboxylate salt comprises an acetate, ascorbate, benzoate, butyrate, citrate, lactate, levulinate, malate, pyruvate, succinate, tartrate, or salicylate salt.

4. The use of claim 1, wherein the synthetic nicotine analogue comprises (S)-6-methylnicotine.

5. The use of claim 1, wherein the synthetic nicotine analogue is used as a nicotine replacement for electronic delivery systems.

6. The use of claim 1, wherein the synthetic nicotine analogue is used as a nicotine replacement in combustion applications.

7. The use of claim 1, wherein the synthetic nicotine analogue is used as a nicotine replacement in heat-not-burn applications.

8. The use of claim 1, wherein the synthetic nicotine analogue is used as a nicotine replacement in oral delivery applications.

9. The use of claim 1, wherein the synthetic nicotine analogue is used as a nicotine replacement in nasal delivery applications.

10. The use of claim 1, wherein the synthetic nicotine analogue is used as a nicotine replacement in heat-not-burn applications.

11. The use of claim 1 for nicotine replacement therapy.

12. The use of claim 1, wherein the synthetic nicotine analogue is a formulation containing between 0.01% and 30.0% (S)-6-methylnicotine or a carboxylate salt thereof.

13. Use of a synthetic nicotine analogue as a nicotine amplification agent, wherein the synthetic nicotine analogue comprises (S)-6-methylnicotine or a carboxylate salt thereof.

14. The use of claim 13, wherein the synthetic nicotine analogue comprises a(S)-6-methylnicotine carboxylate salt.

15. The use of claim 14, wherein the (S)-6-methylnicotine carboxylate salt comprises an acetate, ascorbate, benzoate, butyrate, citrate, lactate, levulinate, malate, pyruvate, succinate, tartrate, or salicylate salt.

16. The use of claim 13, wherein the synthetic nicotine analogue comprises (S)-6-methylnicotine.

17. A synthetic nicotine analogue comprising enantiomerically pure (S)-6-methylnicotine or a carboxylate salt thereof.

18. The synthetic nicotine analogue of claim 17, wherein the carboxylate salt is an acetate, ascorbate, benzoate, butyrate, citrate, lactate, levulinate, malate, pyruvate, succinate, tartrate, or salicylate salt.

19. A synthetic nicotine analogue formulation, comprising between 0.01% and 30.0% of the synthetic nicotine analogue of claim 17.
